(19) United States
(12) Patent Application Publication
(10) Pub. No.: US 2023/0380930 A1
(43) Pub. Date: Nov. 30, 2023

(54) MILLING BUR FOR IMPLANT AND METHOD FOR FORMING IMPLANT HOLE USING THE SAME

(71) Applicant: Chaeheon HEO, Seoul (KR)

(72) Inventor: Chaeheon HEO, Seoul (KR)

(21) Appl. No.: 18/202,466

(22) Filed: May 26, 2023

(30) Foreign Application Priority Data
May 27, 2022 (KR) 10-2022-0065367

(51) Int. Cl. A61C 1/08; A61C 19/04

(52) U.S. Cl. CPC A61C 1/08; A61C 19/04; A61B 34/30

(57) ABSTRACT

A milling bur for an implant that is used to form a hole for implanting an artificial tooth is formed in a stick shape, has a shape of which the diameter decreases toward an end from an upper end that is coupled to an operating robot, has a milling blade for forming a hole for implanting an artificial tooth in an alveolar bone at the end, and is formed such that the diameter of the milling blade is larger than the diameter of the end.

### CROSS REFERENCE TO RELATED APPLICATION AND CLAIM OF PRIORITY

This application claims the benefit under 35 USC § 119 of Korean Patent Application No. 10-2022-0065367, filed on May 27, 2022, in the Korean Intellectual Property Office, the entire disclosure of which is incorporated herein by reference for all purposes.

### BACKGROUND

### 1. Field of the Invention

The present disclosure relates to a milling bur for an implant and a method for forming an implant hole that forms an implant hole in the alveolar bone.

### 2. Description of the Related Art

Teeth are very important for human, and when teeth are pulled out due to damage to the teeth, it is difficult to eat food, which results in damage in terms of nutrition.

Accordingly, artificial teeth are formed through prostheses using undamaged teeth as supports when one or two teeth are pulled out, or a denture fitting to the gum and oral cavity is used when most teeth are damaged and pulled out.

However, as for a prosthesis that uses an artificial tooth, an artificial tooth is supported by undamaged teeth, so it is relatively difficult to eat hard food and a sense of foreign body is caused. Further, when the shape of a denture and the shape of an oral cavity are not the same in the case of using a denture, users feels discomfort and the oral cavity may be injured by the denture. Further, it is difficult to eat hard food and, above all, there is a trouble that users have to take out and wash or sterilize a denture before going to bed at night.

As a measure for solving these problems, a dental implant surgery that forms a hole in the alveolar bone with a tooth missing and then embeds an artificial tooth having threads on the lower portion into the alveolar bone has been proposed.

A dental implant surgery is, as shown in FIG. 1, a procedure of implanting an artificial tooth by making a hole in the alveolar with a tooth missing at an appropriate depth for implanting an artificial tooth using a medical drill and then by inserting and fixing an artificial tooth with a screw on the lower portion into the hole.

In general, the alveolar is composed of a supporting alveolar bone positioned outside and an alveolar bone proper positioned inside the supporting alveolar bone, and the supporting alveolar bone is harder than alveolar bone proper. Further, the people have different bone densities of alveolar bone proper and the bone densities may change depending on the depth direction.

It is important to bore a hole with a diameter suitable for an artificial tooth when boring a hole for implanting an artificial teeth in the alveolar bone using a medical drill, and it is required to appropriately adjust the diameter of a hole in order to firmly fix an implanted artificial tooth and the alveolar bone to each other. However, since a hole formed by a medical drill is almost the same in diameter as the drill, it is required to prepare drills with various diameters to correspond to holes with various sizes. Further, friction heat is generated in boring by drill, so saline solution is also supplied to reduce such friction heat. However, since the side of a drill and the wall of a bored alveolar bone are almost in close contact with each other, heat may not be appropriately removed due to poor supply of saline solution.

Accordingly, it may be preferable to provide a device that can efficiently remove heat, which is generated in boring, while using a small number of parts and that can form a 3D hole in the alveolar bone, and a boring method using the device.

### SUMMARY

An objective of the present disclosure is to provide a milling bur for an implant that can form holes having various diameters in the depth direction in the alveolar bone.

Another objective of the present disclosure is to provide a method for forming a 3D hole for implanting an artificial tooth in the alveolar bone using the milling bur for an implant.

A milling bur for an implant that is used to form a hole for implanting an artificial tooth is formed in a stick shape, has a shape of which the diameter decreases toward an end from an upper end that is coupled to an operating robot, has a milling blade for forming a hole for implanting an artificial tooth in an alveolar bone at the end, and is formed such that the diameter of the milling blade is larger than the diameter of the end.

A scale that makes it possible to check a depth of an implant hole is formed over an end of the milling bur for an implant.

In the milling bur for an implant, the milling blade is formed in a cylindrical shape having a rectangular cross-section, cutting blades for cutting an alveolar bone and cutting grooves for discharging cut bone fragments are formed on an outer surface of the milling blade, and the cutting blades and the cutting grooves extend to a bottom of the milling blade.

In the milling bur for an implant, the milling blade is formed to have a circular, inverted triangular, or spiral cross-section, cutting blades for cutting an alveolar bone and cutting grooves for discharging cut bone fragments are formed on an outer surface of the milling blade, and the cutting blades and the cutting grooves extend to a bottom of the milling blade.

In the milling bur for an implant, the milling blade is formed to have a rectangular, circular, or inverted triangular cross-section, an outer surface of the milling blade is flat, and diamond powder for cutting an alveolar bone is fixed on the outer surface.

The milling bur for an implant is mounted on an operating robot, the operating robot forms a hole in an alveolar bone by driving the milling bur up and down, and left and right in a circular shape, and a diameter of the milling blade of the milling bur is smaller than a diameter of the hole.

A method for forming an implant hole using the milling bur includes: mounting the milling bur on an operating robot; measuring bone density of an alveolar bone using a device for measuring bone density; storing data of the measured bone density in the operating robot; and forming a hole in an alveolar bone by holding the milling bur, moving the milling blade of the milling bur to the alveolar bone to bore, and moving the milling bur in a depth direction of the alveolar bone while moving the milling bur left and right in a circular shape by means of an operator to form a hole in an alveolar bone.

In the method for forming an implant hole using the milling bur for an implant, a portion with high bone density is cut to shallowly come in contact with a screw of an artificial tooth and a portion with low bone density is cut to deeply come in contact with the screw so that the screw of the artificial tooth that is installed in the hole has a uniform contact force with the alveolar bone.

In the method for forming an implant hole, a hole is formed to have various diameters in accordance with bone density of the alveolar bone.

### BRIEF DESCRIPTION OF THE DRAWINGS

FIG. 1 is a view showing a drill that is used in a dental implant surgery in the related art;

FIGS. 2A and 2B are a view showing the configuration of a milling bur according to the present disclosure;

FIGS. 3A and 3B are a view comparing defects in use of an existing drill and advantages of the milling bur according to the present disclosure depending on the shapes of alveolar bones;

FIGS. 4A to 4C are a view showing formation of a hole by an existing drill and formation of a hole by the milling bur of the present disclosure; and

FIG. 5 is a view showing a method for forming an asymmetric hole under an extreme bone density to describe the present disclosure in more detail.

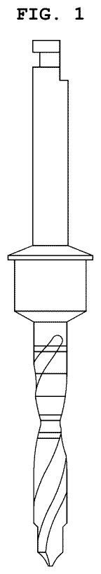

### DETAILED DESCRIPTION

Hereinafter, exemplary embodiments of the present disclosure will be described in more detail with reference to accompanying drawings.

Before describing the present disclosure, specific structures and functions are exemplified only to describe embodiments according to the concept of the present disclosure, and embodiments according to the concept of the present disclosure may be achieved in various ways and should not be construed as being limited to the embodiments described herein.

Further, embodiments described herein may be changed in various ways and may have various shapes, so specific embodiments are shown in the drawings and will be described in detail in this specification. However, it should be understood that the exemplary embodiments according to the concept of the present disclosure are not limited to the embodiments which will be described hereinbelow with reference to the accompanying drawings, but all modifications, equivalents, and substitutions are included in the scope and spirit of the present disclosure.

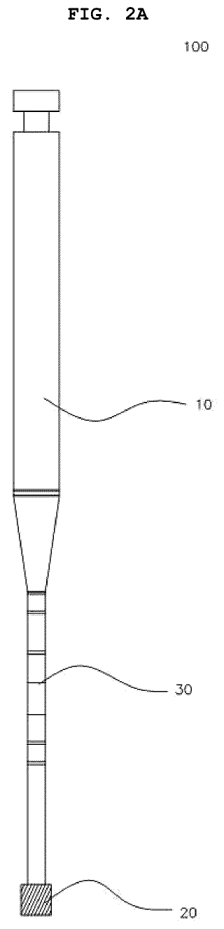

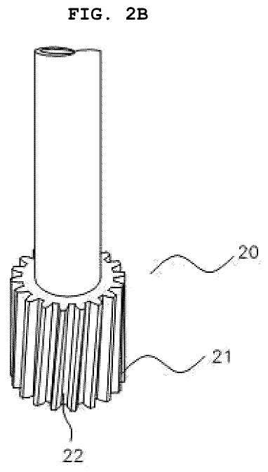

FIGS. 2A and 2B are a view showing a milling bur for an implant according to the present disclosure. FIG. 2A is a view showing the side of the milling bur and FIG. 2B is an enlarged view of a milling blade.

As shown in FIG. 2A, a milling bur 100 according to the present disclosure is formed in a stick shape and includes an upper part 10 that is fastened to an operating robot (not shown) and is held by an operator, and a milling blade 20 that is installed at a second end of a supporting rod, of which a first end is connected to an end of the upper part, to form a hole for implanting an artificial tooth in the alveolar bone for a dental implant surgery. The diameter of the milling blade is larger than the diameter of the supporting rod. Further, since the supporting rod is a part that is inserted in an implant hole when the hole is formed, the supporting rod is formed generally longer than the depth of an implant hole. In general, it is preferable that the length is 15 mm to 20 mm.

Further, a scale 30 that makes it possible to check the depth of an implant hole is formed at a predetermined position on the supporting rod to which the milling blade is connected.

As shown in FIG. 2B, the milling blade 20 is formed in a cylindrical shape having a rectangular cross-section in the figure, but may have a circular or inverted triangular cross-section, depending on implant surgeries. Meanwhile, in order to form a hole in the alveolar bone and form threads for fastening to the screw of an implant at the same time, the milling blade may be manufactured in a disc shape and cutting blades are formed on the side and the bottom. Accordingly, it is possible to form not only a hole in the alveolar bone, but also threads in the formed hole.

Cutting blades 21 for cutting the alveolar bone and cutting grooves 22 for discharging cut bone fragments are formed on the outer surface of the milling blade 20. The cutting blades and the cutting grooves extend to the bottom of the milling blade.

However, it may be possible to attach fine industrial diamond powder to the outer surface of the milling blade and perform cutting without forming cutting blades and cutting grooves.

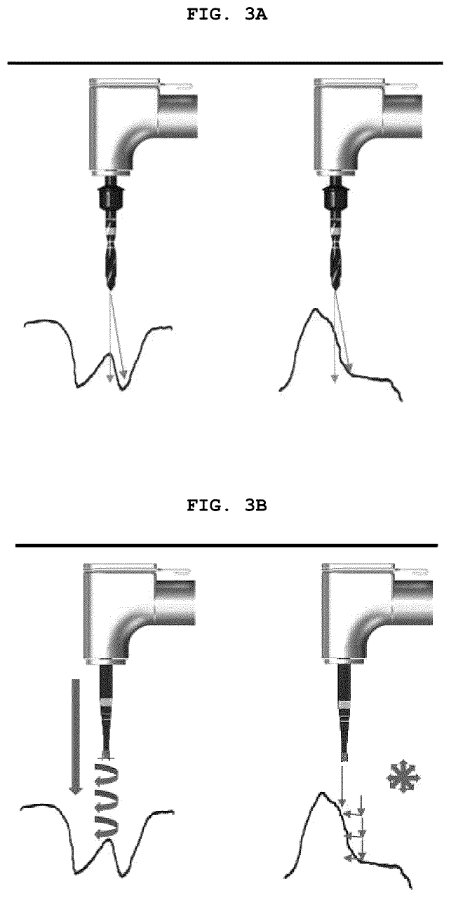

FIGS. 3A and 3B show problems that may be generated when boring a hole for implanting an artificial tooth in the alveolar bone using an implant drill of the related art.

As shown in FIG. 3A, protrusions were formed at the middle potion of the alveolar bone in many cases. In these case, when a hole for implanting an artificial tooth is bored using an implant drill of the related art, the progress direction of the drill may unexpectedly finely change when the drill hits against the side of a protrusion. When this situation occurs, a hole for implanting an artificial tooth is not accurately formed, whereby an artificial tooth may be not completely implanted. In particular, when several artificial teeth are implanted, the implanted artificial teeth may interfere with each other.

However, as shown in FIG. 3B, when the milling bur according to the present disclosure is used, the milling blade at the end of the milling bur cuts not only the bottom, but also the side of the alveolar bone, so even if the milling blade hits against the side of a protrusion, it can accurately vertically form a hole without changing the progress direction.

Accordingly, an artificial tooth is accurately and precisely implanted by using the milling bur according to the present disclosure.

As described above, the alveolar bone is composed of a compact bone and a cancellous bone positioned inside the compact bone. The compact bone has relatively high hardness because the bone density is considerably high, but the bone density of the cancellous bone is lower than that of the compact bone and decreases as it goes to the jaw from the top. However, this is a general knowledge, and the cancellous bone may have various bone densities in the depth direction depending on people.

When a hole is formed in the alveolar bones having various bone densities, as described above, using a common implant drill, the hole has a constant diameter. The diameter of a hole is determined smaller than the diameter of the screw of an artificial tooth to firmly fix the artificial tooth. However, the alveolar bone having high bone density is in strong contact with the screw of an artificial tooth under excessive pressure, but the alveolar bone having low bone density is relatively in loose contact with the screw of an artificial tooth.

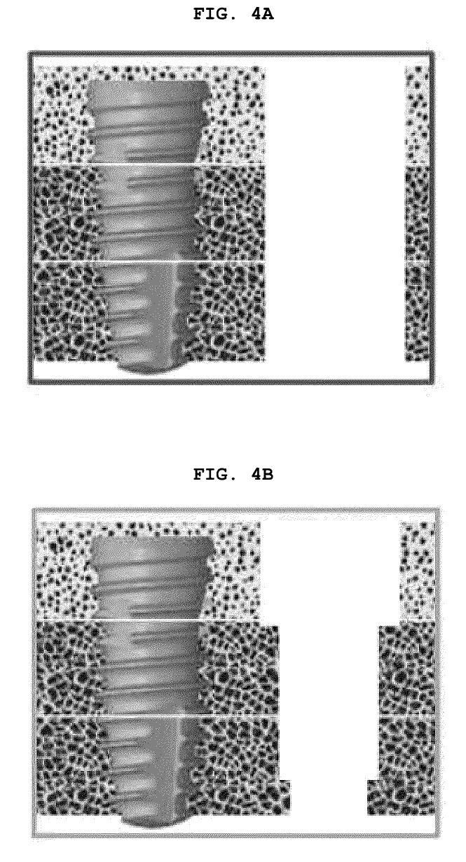

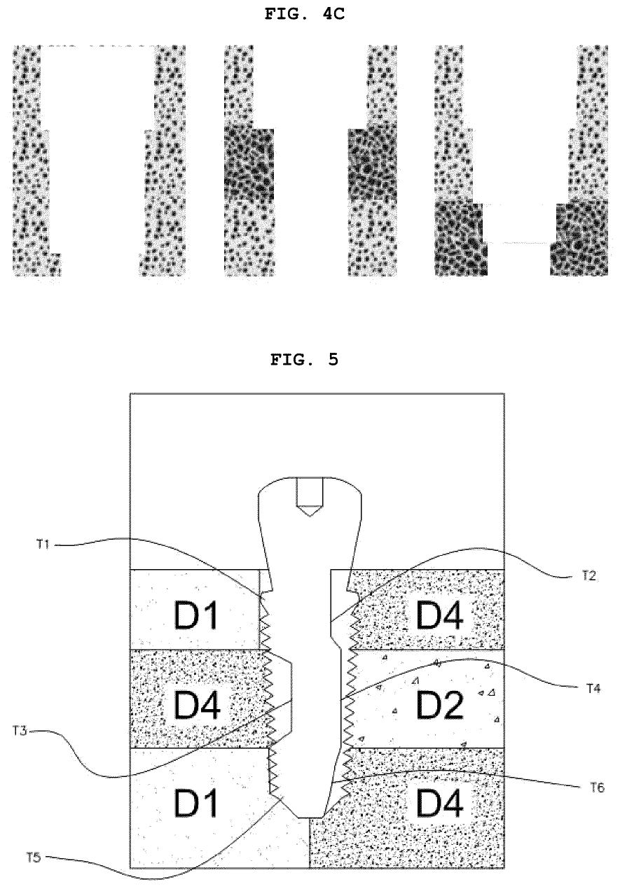

FIG. 4A is a view showing that a hole for an artificial tooth is formed using an existing implant drill and the screw of an artificial tooth is coupled to the hole. In this figure, the light color means that the bone density of the alveolar bone is relatively high, and it shows that the deeper the color, the higher the bone density.

As shown in FIG. 4A, a hole for an artificial tooth that is formed using an existing implant drill is formed straightly and has the same diameter as the drill. Further, the screw of an artificial tooth is formed such that the diameter decreases as it goes down, as shown in the figure. Accordingly, when the artificial screw of an artificial tooth is inserted into the hole formed by an existing drill, the relatively large-diameter section of the screw comes in contact with the upper portion with high bone density, so a strong contact force is generated; however, the relatively small-diameter section of the screw comes in contact with the lower portion with low bone density, so the contact force is relatively small. Accordingly, the screw of the artificial tooth may not be firmly fixed in the alveolar bone.

Further, when the diameter of a hole is decreased to increase a contact force at the lower portion with low bone density, an excessive contact force with a screw may be generated at the upper portion with high bone density and the excessive contact force may necrose the alveolar bone.

FIG. 4B shows a hole formed in the alveolar bone using a milling bur for an implant according to the present disclosure.

As described above, existing implant drills and the milling bur according to the present disclosure are coupled to an operating robot to operate. Existing drills can be moved only up and down, that is, two-dimensionally by an operating robot, but the milling bur according to the present disclosure can be moved not only up and down, but also left and right in a circular shape, that is, three-dimensionally. Further, the milling bur according to the present disclosure has a lateral cutting function unlike existing drills.

As shown in FIG. 4B, unlike forming a hole in the alveolar bone in correspondence to the diameter of a drill using a drill that can be only two-dimensionally moved, the milling bur according to the present disclosure forms a hole in the alveolar bone through three-dimensional movement, so the milling blade of the milling bur has a diameter corresponding to or smaller than a minimum-diameter hole to be formed. The milling bur with a milling blade having this diameter is mounted on an operating robot, cuts the alveolar bone to have a relatively large diameter at the upper portion with high bone density, and cuts the alveolar bone to have a relatively small diameter as it goes down to the lower portion with low bone density. Accordingly, the cross-section of the formed hole has the shape shown in FIG. 4B.

Accordingly, when the screw of an artificial tooth is inserted into the hole, the screw is fixed by a uniform and high contact force at both the upper and lower portions of the alveolar bone. Therefore, high stability is achieved from the early stage of implantation of an artificial tooth, as compared with implanting an artificial tooth using existing drills.

FIG. 4C shows an example of a hole formed in accordance with bone density of the alveolar bone. As shown in the figure, it can be seen that the size of a hole formed in accordance with the bone density of the alveolar is various.

Meanwhile, there is a problem that it is required to replace drills having diameters corresponding to the diameters of holes to be formed in order to form holes having various diameters in the alveolar bone using existing implant drills. However, in the present disclosure, since a milling blade having a diameter smaller than the diameter of a hole to be formed is driven up, down, left, and right, that is, three-dimensionally using an operating robot, there is an advantage that it is possible to form holes having various sizes using one milling bur regardless of the sizes of holes to be formed in the alveolar bone. That is, there is an advantage that it is possible to remarkably reduce the number of parts, that is, tools for forming holes in the alveolar bone.

FIG. 5 is a view showing an example of forming an asymmetric hole in the alveolar bone having extreme bone density and installing a screw, in which D1 indicates a region with the highest bone density, D2 indicates a region with a next high bone density, and D4 indicates a region with the lowest bone density.

As shown in FIG. 5, the region D1 having the highest bone density is bored such that the surface that comes in contact with a screw is low (T1, T5), the region D2 with the next high bone density is bored to come in contact with a screw more than the region D1 (T4), and the region D4 with the lowest bone density is bored to come in contact with a screw the most (T2, T3, T6). In this case, a hole is milled to be smaller than the diameter of the screw of an implant such that the screw of the implant itself cuts the alveolar, thereby being able to secure an additional fixing force.

By forming a hole and inserting a screw in the hole in this way, a uniform and strong contact force is maintained throughout the contact surface of the screw and it is possible to prevent necrosis of the alveolar due to excessive contact pressure.

The method for forming a hole for an implant in the alveolar bone using the milling bur according to the present disclosure is described again. First, the bone density of the alveolar bone is measured. Bone density can be measured through devices such as a CT system. Next, the measured bone density is stored in an operating robot and the milling bur according to the present disclosure is mounted on the operating robot. Next, an operator who is a dentist holds the milling bur, moves the milling blade of the milling bur to the alveolar bone to bore, and moves the milling bur in the depth direction of the alveolar bone while moving it left and right in a circular shape, thereby forming a hole in the alveolar bone.

In this case, when the range limit of the milling bur according to the bone density stored in the operating robot is exceeded, the operator is warned by a haptic function of the milling bur. However, the information of bone density stored in an operating robot and the information of the actual bone density may be different, and in this case, there is a risk of excessive or insufficient cutting of the alveolar bone. In order to remove this risk, it may be possible to install a sensor, which can measure changes in torque that is applied to the milling bur, on an operating robot on which the milling bur is mounted. Accordingly, it is possible to safely form a hole initially intended by operating the milling bur on the basis of the information of bone density stored in an operating robot and a torque value that is measured in real time.

Various matters are described in detail in the above description, but these should be construed as an example of a preferred embodiment rather than limiting the range of the present disclosure. Accordingly, the present disclosure should be determined by claims and equivalents to the claims rather than being determined by the embodiment described above.

According to the milling bur for an implant of the present disclosure and the method for forming an implant hole using the milling bur, since it is possible to respond to holes having various diameters using one milling bur, there is an effect that it is possible to reduce the number of tools for a dental implant surgery.

Further, according to the milling bur of the present disclosure, there is an effect that it is possible to form a hole having various diameters at a time in accordance with the bone density of the alveolar bone and it is possible to achieve a uniform and strong contact force between the alveolar bone and the screw of an implant by adjusting the diameter of a hole in accordance with bone density.

While the present invention has been described with respect to the specific embodiments, it will be apparent to those skilled in the art that various changes and modifications may be made without departing from the spirit and scope of the invention as defined in the following claims.

What is claimed is:

1. A milling bur configured to be used to form a hole for implanting an artificial tooth, the milling bur comprising:
an upper part configured to be fastened to an operating robot and configured to be held by an operator;
a supporting rod having a first end connected to an end of the upper part and a second end; and
a milling blade installed at the second end of the supporting rod, the milling blade configured to form a hole for implanting the artificial tooth in an alveolar bone for a dental implant surgery, a diameter of the milling blade being larger than a diameter of the supporting rod, wherein the milling bur is formed in a stick shape.

2. The milling bur of claim 1, wherein the supporting rod has a scale formed thereon configured to check a depth of the hole.

3. The milling bur of claim 1, wherein the milling blade is formed in a cylindrical shape having a rectangular cross-section;
the milling blade has an outer surface on which cutting blades for cutting an alveolar bone and cutting grooves for discharging cut bone fragments are formed; and
the cutting blades and the cutting grooves extend to a bottom of the milling blade.

4. The milling bur of claim 3, wherein the milling bur is configured to be mounted on the operating robot;
the operating robot is configured to form the hole in an alveolar bone by driving the milling bur up and down, and left and right in a circular shape; and
a diameter of the milling bur is smaller than a diameter of the hole.

5. The milling bur of claim 1, wherein the milling blade has a circular or inverted triangular cross-section;
the milling blade has an outer surface on which cutting blades for cutting an alveolar bone and cutting grooves for discharging cut bone fragments are formed; and
the cutting blades and the cutting grooves extend to a bottom of the milling blade.

6. The milling bur of claim 5, wherein the milling bur is configured to be mounted on the operating robot;
the operating robot is configured to form the hole in an alveolar bone by driving the milling bur up and down, and left and right in a circular shape; and
a diameter of the milling bur is smaller than a diameter of the hole.

7. The milling bur of claim 1, wherein the milling blade has a rectangular, circular, or inverted triangular cross-section;
an outer surface of the milling blade is flat; and
diamond powder for cutting an alveolar bone is fixed on the outer surface.

8. The milling bur of claim 7, wherein the milling bur is configured to be mounted on the operating robot;
the operating robot is configured to form the hole in an alveolar bone by driving the milling bur up and down, and left and right in a circular shape; and
a diameter of the milling bur is smaller than a diameter of the hole.

9. A method for forming a hole for implanting, the method comprising:
mounting the milling bur of claim 1 on the operating robot;
measuring bone density of an alveolar bone using a device for measuring bone density;
storing data of the measured bone density in the operating robot; and
forming the hole in an alveolar bone by holding the milling bur, moving the milling blade of the milling bur to the alveolar bone to bore, and moving the milling bur in a depth direction of the alveolar bone while moving the milling bur left and right in a circular shape by the operator.

10. The method for claim 9, wherein the hole is asymmetrically cut so that a portion with high bone density shallowly comes in contact with a screw of an artificial tooth and a portion with low bone density deeply comes in contact with the screw so that the screw of the artificial tooth that is installed in the hole has a uniform contact force with the alveolar bone.

11. The method for claim 10, wherein the hole is asymmetrically formed to have various diameters in accordance with bone density of the alveolar bone.

12. The method for claim 10, wherein the milling blade has a disc shape;
the milling blade has an outer surface on which cutting blades for cutting an alveolar bone and cutting grooves for discharging cut bone fragments are formed; and
the cutting blades and the cutting grooves extend to a bottom of the milling blade.

13. The method for claim 12, wherein the milling bur forms a hole in an alveolar bone, and form threads for coupling to a screw of an implant in the hole.
